(19) United States
(12) Patent Application Publication
(10) Pub. No.: US 2007/0032665 A1
(43) Pub. Date: Feb. 8, 2007

(54) PREPARATION OF ATORVASTATIN CALCIUM FORM I

(76) Inventors: Srinivasulu Gudipati, Hyderabad 500 072, A.P. (IN); Srinivas Katkam, Secundarabad 500 047, A.P. (IN); Satyanarayana Komati, Hyderabad 502 032, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/462,084

(22) Filed: Aug. 3, 2006

Related U.S. Application Data
(60) Provisional application No. 60/766,254, filed on Jan. 5, 2006.

(30) Foreign Application Priority Data
Aug. 4, 2005 (IN) 1062/CHE/2005

(51) Int. Cl. C07D 207/24

(52) U.S. Cl. 548/537

(57) ABSTRACT

A process for preparing atorvastatin calcium Form I, comprising adding a solution comprising atorvastatin calcium and an alcohol to water. Optionally, the water can contain seed crystals of atorvastatin calcium Form I.

### INTRODUCTION TO THE INVENTION

The present invention relates to a process for the preparation of atorvastatin calcium crystalline Form I.

Atorvastatin calcium is chemically known as [R-(R*,R*)]-2-(4-fluorophenyl)-β, δ-dihydroxy-5-(1-methylethyl)-3-phenyl-4 [(phenylamino)carbonyl]-IH-pyrrole-1-heptanoic acid, calcium salt (2:1) trihydrate (which is hereinafter referred to by its adopted name “atorvastatin calcium”), and has the structure as represented in Formula I.

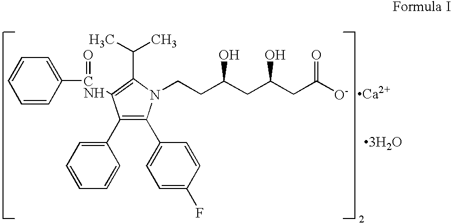

Atorvastatin calcium is a synthetic lipid-lowering agent and is an inhibitor of 3-hydroxy-3-methylglutaryl-coenzyme-A (HMG-CoA) reductase. This enzyme catalyzes the conversion of HMG-COA to mevalonate, an early and rate-limiting step in cholesterol biosynthesis. It is commercially available under the brand name LIPITOR in the form of tablets, which contains 10, 20, or 40 mg atorvastatin

U.S. Pat. No. 5,273,995 discloses atorvastatin and its related compounds along with their pharmaceutically acceptable salts. It also describes pharmaceutical compositions comprising atorvastatin calcium and their use in the treatment of hypercholesterolemia

U.S. Patent No. 5,969,156 discloses atorvastatin calcium crystalline Form I, Form II, Form IV and processes for their preparation.

U.S. Patent Application Publication No. U.S. 2002/0183378 A1 and U.S. 2003/0212279 discloses processes for the preparation of atorvastatin calcium crystalline Form I.

There is a continuing need to prepare stable crystalline forms of active substances such as atorvastatin calcium in an industrially simple and readily feasible way with high yields.

### SUMMARY OF THE INVENTION

The present invention relates to a process for the preparation of atorvastatin calcium crystalline Form I.

In an embodiment the process for the preparation of atorvastatin calcium crystalline Form I comprises:

i) providing a solution of atorvastatin calcium in a suitable solvent; and

ii) adding the solution obtained in step i) to water.

In yet another embodiment the invention provides atorvastatin crystalline Form I substantially free of residual solvents.

In still another embodiment, atorvastatin crystalline Form I prepared according to the present invention has a mean particle size less than about 5 μm.

An aspect of the invention is a process for preparing atorvastatin calcium Form I, comprising adding a solution comprising atorvastatin calcium and an alcohol to water.

Another aspect of the invention is a process for preparing atorvastatin calcium Form I, comprising adding a solution comprising atorvastatin calcium and methanol to water containing seed crystals of atorvastatin calcium Form I.

### BRIEF DESCRIPTION OF THE DRAWINGS

FIG. 1 is an XRPD pattern of the crystalline Form I of atorvastatin calcium prepared according to Example 1.

### DETAILED DESCRIPTION OF THE INVENTION

The present invention relates to a process for the preparation of atorvastatin calcium crystalline Form I.

In an embodiment process for the preparation of atorvastatin calcium crystalline Form I comprises:

i) providing a solution of atorvastatin calcium in a suitable solvent; and

ii) adding the solution obtained in step i) to water.

Step i) involves providing a solution of atorvastatin calcium in a suitable solvent.

The solution of atorvastatin calcium may be obtained by dissolving atorvastatin calcium in a suitable solvent, or such a solution may be obtained directly from a reaction in which atorvastatin calcium is formed

When the solution is prepared by dissolving atorvastatin calcium in a suitable solvent, any form of atorvastatin calcium such as any crystalline or amorphous form, including any salts, solvates and hydrates may be utilized for preparing the solution.

In one embodiment atorvastatin calcium crystalline forms VI and VII are used in the preparation of crystalline form I. Processes for the preparation forms VI and VII are described in WO 03/011826, which is incorporated here in its entirety.

Suitable solvents which can be used in the process of step i) include, but are not limited to, water miscible solvents such as alcohols, including methanol, ethanol, isopropyl alcohol, n-butanol, tertiary butyl alcohol and the like. Of course, any solvent or mixture of solvents in any ratio is acceptable as long as it provides required solubility.

The solution obtained in step i) can be optionally treated with activated charcoal to enhance the color of the compound followed by filtration through an inert medium such as flux calcined diatomaceous earth (Hyflow) bed to remove the carbon or by using other clarification techniques known to those skilled in the art.

The concentration of the solution can be about 50 g/l to about 400 g/l of the solvent. Any other concentration may be used as long as a clear solution is obtained, and the maximum solubility will, of course, differ according to the particular solvent that is being used.

Suitable temperatures for preparing the solution of atorvastatin calcium may range form about 0 to 20° C., or about 0 to 40° C., or higher.

Depending upon the equipment used and the concentration and temperature of the solution, the filtration apparatus may need to be preheated to avoid premature crystallization. Those skilled in the art are aware of various alternative methods for solid-liquid separation, such as decantation, centrifugation, and others.

Step ii) involves adding the solution obtained in step i) to water;

Optionally, small amounts of seeding crystals of atorvastatin calcium Form I may be added to the mixture. Preferably, small amounts are about 0.1 to 20 weight %, or about 0.2 to 0.5 weight %, of the water used. Seeding crystals may be added before or, where appropriate, after the step initiating the precipitation. Seed crystals will typically have sizes somewhat smaller than the desired product particle sizes.

Suitable temperatures for addition of the solution to water may range from about 20° C. to about 70° C. Useful ratios of solvent to the water range from 1:1 to about 1:10 by volume, or about 1:1 to about 1:5, or about 1:1 to about 1:2 by volume.

To enhance crystallization, the reaction mass may be further maintained at temperatures lower than the temperatures at which the solution is added to water, such as for example about 1° C. to about 10° C., or about 10° C. to about 25° C., for a period of time as required for a more complete isolation of the product. The exact cooling temperature and time required for complete crystallization can be readily determined by a person skilled in the art and will also depend on parameters such as concentration and temperature of the solution or slurry.

In all of the above-mentioned processes, the crystallization step can be performed with or without stirring. The crystallization step can require various times, as can easily be determined through simple experiments.

Crystalline Form I of atorvastatin calcium can be recovered from the final mixture, with or without cooling below the crystal formation temperature, can be any of techniques such as filtration by gravity, or by suction, centrifugation, and the like.

The crystals so isolated can carry a small proportion of occluded mother liquor. If desired, the crystals can be washed on the filter with a solvent. The solid can either be washed with the solvent used for dissolution in step i) or using a mixture of solvents used in steps i) and ii).

The wet solid obtained after filtration can optionally be dried under ambient or reduced pressure. For example, drying can be performed under reduced pressure or under atmospheric pressure at a temperature of at about 40° C. to 60° C., or 70° C. to 80° C., or higher, depending on the volatility of the solvent that is being removed. The atmosphere for drying can be air or a partially or completely inert atmosphere, such as by using nitrogen.

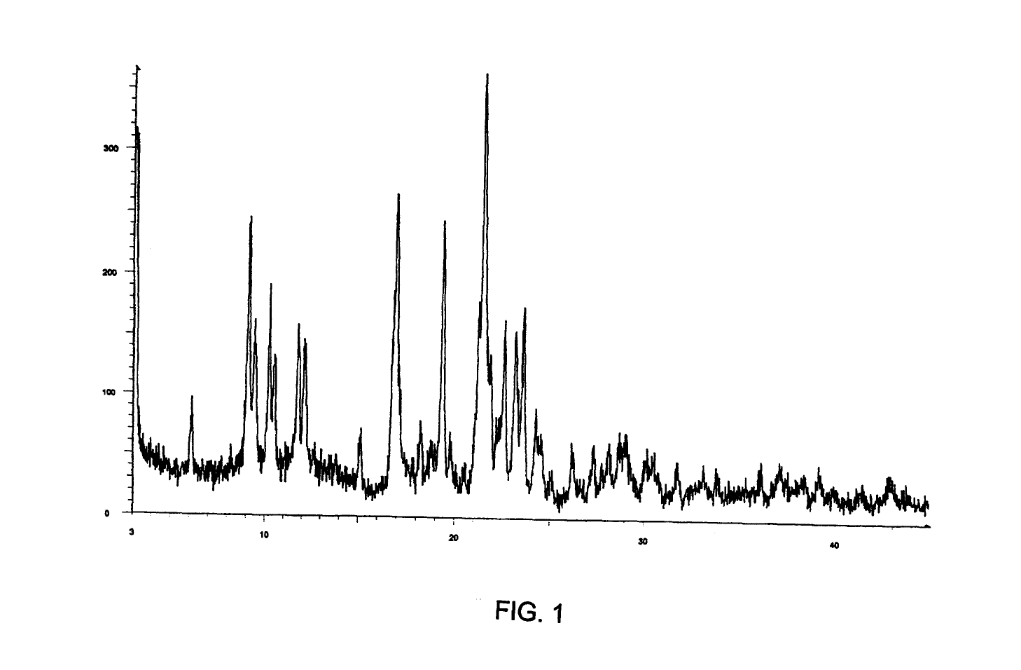

The atorvastatin calcium crystalline Form I prepared according to the present invention is characterized by its diffraction pattern. X-ray powder diffraction (“XRPD”) patterns reported herein were measured on a Bruker Axe, D8 Advance Powder X-ray Diffractometer with a Cu K alpha-1 radiation source and atorvastatin calcium crystalline Form I has an XRPD pattern having significant characteristic peaks at about 9.0, 9.3, 10.1, 10.4, 11.7, 12, 16.9, 19.3, 21.2, 21.4, 21.8, 22.5, 23.1, 23.5, and 42.9, ±0.2° 2θ. An XRPD pattern of the atorvastatin calcium Form I is shown in FIG. 1.

Atorvastatin calcium Form I prepared in this invention contains: less than about 3000 ppm, or less than about 500 ppm, or less than about 50 ppm of methanol; less than about 410 ppm, or less than about 100 ppm, or less than about 50 ppm of acetonitrile; less than about 5000 ppm, or less than about 500 ppm, or less than about 50 ppm of 2-propanol; less than about 3880 ppm, or less than about 500 ppm, or less than about 50 ppm of cyclohexane; less than about 1000 ppm, or less than about 200 ppm, or less than about 50 ppm of tertiary butyl alcohol; and less than about 890 ppm, or less than about 100 ppm, or less than about 50 ppm of toluene. In an embodiment, atorvastatin calcium Form I prepared in this invention is substantially free from the aforementioned residual solvents.

“Substantially free” as used herein means that an individual residual solvent content is less than about 30 ppm in the atorvastatin calcium Form I product. In some instances, it is possible to have less than about 10 ppm of a residual solvent, or a concentration less than or equal to the limit of detection for the analytical method.

In still another embodiment atorvastatin calcium Form I prepared according to the present invention has a mean particle size less than about 5 μm.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using Malvern Instruments Ltd. (of Malvern, Worcestershire, United Kingdom) equipment.

In an embodiment, atorvastatin calcium Form I prepared according to the present invention has a D₁₀ less than 1 μm or less than 0.5 μm, D₅₀ less than 3 μm or less than 2 μm, and D₉₀ less than 10 μm or less than 5 μm. There is no specific lower limit for any of the D values.

The crystalline Form I of atorvastatin calcium of present invention is well suited for use in preparing pharmaceutical formulations.

The process of present invention is cost effective, eco-friendly and commercially suitable, to get stable crystalline Form I of atorvastatin calcium which is free flowing, and directly compressible into stable formulations.

Certain specific aspects and embodiments of this invention are described in further detail by the examples below, which examples are not intended to limit the scope of the appended claims in any manner.

### EXAMPLE 1

Preparation of Atorvastatin Calcium Crystalline Form I Using Form VI

10 g of atorvastatin calcium crystalline Form VI (prepared according to WO 03/011826A1) and 75 ml of methanol were charged into a clean and dry round bottom flask and was stirred for about 15 minutes. 0.5 g of basic activated charcoal was charged and was stirred for about 15 minutes. The resultant reaction suspension was filtered through a celite bed and was washed with 20 ml of methanol. The resultant filtrate was added to a flask containing a mixture of 190 ml water seeded with 0.6 g of pure atorvastatin calcium crystalline Form I, under stirring at about 28° C. over a period of 1.5 hours. The separated solid was filtered and washed with 20 ml of water. The resultant solid was dried at about 65-75° C. for about 60 hours to afford 9.5 g atorvastatin crystalline Form I having an XRPD pattern as shown in FIG. 1, where the vertical axis is intensity and the horizontal axis is the 2θ angle, in degrees.

### EXAMPLE 2

Preparation of Atorvastatin Calcium Crystalline Form I Using Form VII

100 liters of methanol was taken into a reactor and subjected to heating to a temperature of about 35° C. followed by the addition of 31.4 kg of atorvastatin calcium form VII (prepared according to WO 03/011826A1) and subjected to stirring for a period of about 10 minutes. 1 kg of activated carbon was added to the above solution, stirred for 1 minute and the obtained mass was filtered through a leaf filter arranged with a flux calcined diatomaceous earth (Hyflow) bed. The filtrate thus obtained was added into a reactor containing 870 liters of water seeded with 2 kg of atorvastatin calcium crystalline Form I and the mass was maintained at a temperature of 35° C. over a period of 1 hour followed by centrifugation, washing the solid obtained with water and spinning for a period of about 3 hours. The obtained wet material was subjected to aerial drying for a period of about 30 minutes followed by drying in a tray drier at a temperature of about 70° C. for 2 hours and then cooling to a temperature of about 45° C. The solid was transferred into a rotary cone vacuum drier and subjected to drying for a period of about 45 minutes at about 70° C. and again transferred into a tray drier, kept for aerial drying for a period of about 30 minutes followed by drying at a temperature of about 70° C. Finally the obtained solid material was subjected to milling in an air jet mill with a feed rate about 30 kg/hour, followed by sifting through a 40 mesh sieve to afford 29.9 kg of atorvastatin calcium Form I.

Particle size distribution:
D₁₀ less than 1 μm.
D₅₀ less than 2 μm.
D₉₀ less than 5 μm.

Residual solvent analysis by gas chromatography:
Methanol: Not detected (≦10 ppm)
Acetonitrile: Not detected (≦30 ppm)
2-propanol: Not detected (≦10 ppm)
Cyclohexane: Not detected (≦30 ppm)
Tertiary butyl alcohol: Not detected (≦30 ppm)
Toluene: Not detected (≦10 ppm)

Moisture content: 4.6 weight % by Karl Fischer.

1. A process for preparing atorvastatin calcium Form I, comprising adding a solution comprising atorvastatin calcium and an alcohol to water.

2. The process of claim 1, wherein an alcohol comprises methanol.

3. The process of claim 1, wherein a solution has an atorvastatin calcium concentration about 50 to about 400 g/l.

4. The process of claim 1, wherein water contains seed crystals of atorvastatin calcium Form I.

5. The process of claim 1, wherein water contains about 0.1 to about 20 weight percent of seed crystals of atorvastatin calcium Form I.

6. The process of claim 1, wherein water contains about 0.2 to about 0.5 weight percent of seed crystals of atorvastatin calcium Form I.

7. The process of claim 1, wherein a ratio of alcohol to water is about 1:1 to about 1:10 by volume.

8. The process of claim 1, wherein a ratio of alcohol to water is about 1:1 to about 1:5 by volume.

9. The process of claim 1, wherein a ratio of alcohol to water is about 1:1 to about 1:2 by volume.

10. The process of claim 1, wherein atorvastatin calcium Form I has a mean particle size less than about 5 μm.

11. Atorvastatin calcium Form I prepared by the process of claim 1, having a particle size distribution of D₁₀ less than about 1 μm, D₅₀ less than about 3 μm, and D₉₀ less than about 10 μm.

12. Atorvastatin calcium Form I prepared by the process of claim 1, being substantially free from residual solvents.

13. A process for preparing atorvastatin calcium Form I, comprising adding a solution comprising atorvastatin calcium and methanol to water containing seed crystals of atorvastatin calcium Form I.

14. The process of claim 13, wherein a solution has an atorvastatin calcium concentration about 50 to about 400 g/l.

15. The process of claim 13, wherein a ratio of methanol to water is about 1:1 to about 1:10 by volume.

16. The process of claim 13, wherein atorvastatin calcium Form I has a mean particle size less than about 5 μm.

17. Atorvastatin calcium Form I prepared by the process of claim 13, having a particle size distribution of D₁₀ less than about 1 μm, D₅₀ less than about 3 μm, and D₉₀ less than about 10 μm.

18. Atorvastatin calcium Form I prepared by the process of claim 13, being substantially free from residual solvents.
